(19) United States
(12) Patent Application Publication
(10) Pub. No.: US 2020/0178911 A1
(43) Pub. Date: Jun. 11, 2020

(54) SYSTEM FOR ENHANCING IMAGE QUALITY OF A SUBJECT USING CBCT (CONE BEAM COMPUTED TOMOGRAPHY)

(71) Applicant: Panacea Medical Technologies Private Limited, Bangalore (IN)

(72) Inventors: Tamilarasan Mani, Banagalore (IN); Goteti Venkata Subrahmanyam, Banagalore (IN)

(21) Appl. No.: 16/634,316

(22) PCT Filed: Jul. 25, 2018
(86) PCT No.: PCT/IB2018/055536

§ 371 Date: Jan. 27, 2020

(30) Foreign Application Priority Data
Jul. 25, 2017 (IN) 201741026459

(51) Int. Cl. A61B 6/03; A61B 6/00

(52) U.S. Cl. CPC A61B 6/032; A61B 6/4447; A61B 6/4085

(57) ABSTRACT

The present invention provides a system (100) for enhancement of the image quality of a subject with less radiation exposure and high spatial resolution using CBCT technique. For this purpose, the system (100) comprises of a high resolution movable Flat Panel Imager (101), an X-Ray Tube (102) which are mounted on the rotatable Gantry (103). The cone beam X-Ray Tube (102) is mounted on opposite side of Flat Panel Imager (101). The entire Gantry (103) is supported with Base Structure (104) along with an angle of +15 degrees or −15 degrees tilting motion. The Patient Couch Table (105) is mounted on the floor based on the gantry reference plane.

### CROSS-REFERENCE TO RELATED APPLICATIONS

The present application is a national phase application and claims priority from PCT application serial no. PCT/IB2018/055536, filed Jul. 25, 2018, which claims priority to Indian patent application serial no. IN 201741026459, filed Jul. 25, 2017, each herein incorporated by reference in their entireties.

### DESCRIPTION OF THE INVENTION

### Technical Field of the Invention

The present invention relates to the field of medical imaging techniques where x-ray source detector system is used to generate scan of entire region of interest using CBCT (Cone Beam Computed Tomography) technique.

### Background of the Invention

Imaging systems are used in a wide variety of medical applications. Existing medical imaging systems rely on Conventional CT (Computed Tomography) devices or scanners in which multiple slices must be stacked to obtain a complete image. Conventional CT scanners employ the use of 1-D (one dimensional) detectors to generate an entire scan of the region of interest.

In another system, conventional fan beam spiral scan geometries are employed in x-ray use. Fan beam scans are obtained by transmission of the FOV (Field of View) by a narrow, fan-shaped beam of x-rays or multiple beams simultaneously. The x-ray beam generated by the tube is collimated to a fan shaped beam by rejecting the photons outside the beam which results in a relatively inefficient use of the x-ray photons.

These are examples from a plethora of medical imaging systems used to obtain a complete image of the inside of the body or any region of interest mentioned herein.

Hence, there exists a need for an advanced medical imaging system which provides complete enhanced images of the inside of the body or any other region of interest such as body tissues, critical structures with reduction in the radiation dosage to the body and increase in the spatial resolution for an improved contrast of the image to be obtained.

According to an aspect of the present invention, an imaging system is provided, particularly for use in medical interventions such as radiology. The imaging system of the invention comprises of CBCT (Cone Beam Computed Tomography) scanners with a flat panel detector representing the next generation of imaging technology available in the interventional radiology suite and is predicted to be the platform for many of the 3-D (three dimensional) road mapping and navigational tools that will emerge in parallel with its integration. The CBCT scanners use a 2-D (two dimensional) detector which allows for a single rotation of the x-ray source detector system to generate a scan of the entire region of interest. In comparison with conventional fan beam spiral scan geometries, cone beam geometry has higher efficiency in x-ray use.

### SUMMARY OF THE INVENTION

The present invention overcomes the drawbacks in the prior art and provides a system for enhancing image quality of a subject using CBCT (Cone Beam Computed Tomography).

The movable flat panel imager is mounted on the opposite side of the X-Ray Tube. The flat panel imager is used along with Cone Beam Computed Tomography (CBCT) to obtain superior image quality. Each projection on the movable flat panel imager is used to image all the organs of the patient body in a single rotation.

The Flat Panel Cone Beam Computed Tomography (FPCBCT) is used to obtain greater field of view (FOV) of the subject. The FPBCT represents 45 cm diameter and a 24 cm axial length cooperatively with the patient that can be imaged with a single 360 degrees rotation of the gantry.

The present invention provides a medical imaging system that enhances the image quality of a subject using CBCT technique

### BRIEF DESCRIPTION OF THE DRAWINGS

The foregoing and other features of embodiments will become more apparent from the following detailed description of embodiments when read in conjunction with the accompanying drawings. In the drawings, like reference numerals refer to like elements.

FIG. 1 illustrates arrangement of components in order to enhance image quality of a subject using CBCT technique in accordance to one embodiment of the present invention.

FIG. 2 illustrates the arrangement of components in order to obtain greater FOV of the subject using FPCBCT (Flat Panel Cone Beam Computed Tomography) in accordance to one embodiment of the present invention.

### DETAILED DESCRIPTION OF THE INVENTION

Reference will now be made in detail to the description of the present subject matter, one or more examples of which are shown in figures. Each example is provided to explain the subject matter and not a limitation. Various changes and modifications obvious to one skilled in the art to which the invention pertains are deemed to be within the spirit, scope and contemplation of the invention.

The present invention overcomes the drawbacks of the prior art by providing a medical imaging system that enhances the image quality of a subject using CBCT technique. For this purpose, the CBCT scanners use a two-dimensional detector which allows a single rotation of the x-ray source detector system to generate a scan of the entire region of interest. The images obtained using CBCT technique cover large volumes and have higher spatial resolution. Scan time is quite less in FPCBCT machines over other CT machines such as multi-detector CT. FPCBCT allows visualization of fine details such as trabecular bone architecture. The CBCT medical imaging system provides reduced radiation exposure. The system comprises of a rotatable gantry in the form of a ring which is mechanically stabled with integrated imaging unit. An integrated bow-tie filter is used for avoiding the scatter radiation.

A flat panel detector is used along with CBCT to obtain superior image quality. Each projection on the flat panel detector is comparable to a standard plain film X-ray and entire organs can be imaged in a single rotation.

The combination of current and unappreciated capabilities may be the foundation on which improvements in both safety and effectiveness of procedures become possible. These improvements include multi-planar soft tissue imaging, enhanced pre-treatment target lesion road mapping and guidance and the ability for immediate multi planar post treatment assessment.

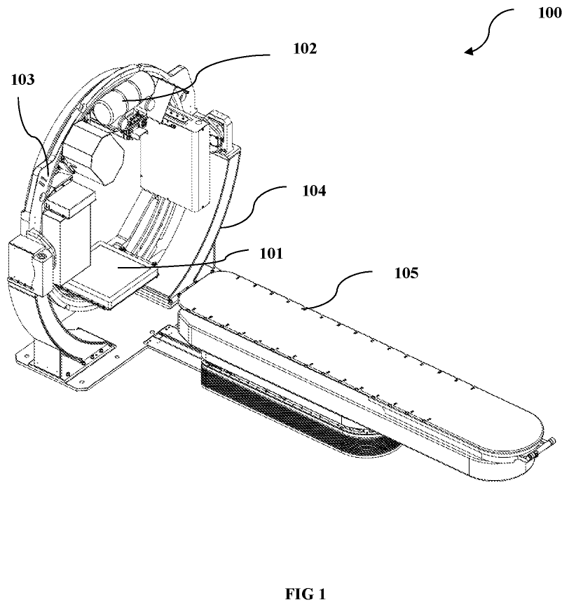

FIG. 1 illustrates the arrangement of components for enhancement of image quality of a subject using CBCT technique in accordance to one or more embodiment of the present invention. The system (100) of the present invention mainly comprises of a Flat Panel (101), an X-Ray Tube (102), Gantry (103), a Base Structure (104) and a Patient Table (105). The gantry (103) is mechanically stabled ring with integrated imaging unit. Continuous rotation of gantry (103) allows depiction of dynamic process such as blood flow. The Patient Table (105) which is a part of the present invention is used for spiral scan. The FPCBCT identifies soft tissue targets and the images obtained also have reduced metal and beam hardening artifacts. An X-Ray Tube or collimator (102) is precisely adjusted for reducing the radiation penumbra.

In accordance to one embodiment of the present invention, the Flat Panel Imager (101) and an X-Ray Tube (102) of the system (100) are mounted on the rotatable gantry (103). The cone beam X-Ray Tube (102) has been mounted on opposite side of Flat Panel Imager (101). This design reduces the misalignment between the flat panel imager (101) to X-Ray Tube (102). The Flat Panel X-Ray detector system provides a low dose CBCT image.

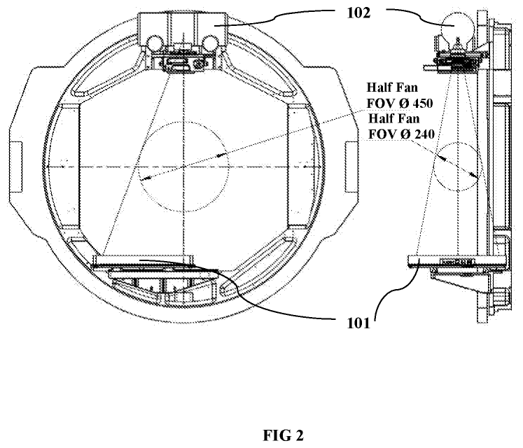

FIG. 2 illustrates components arrangement in order to achieve or obtain greater FOV of the subject using FPCBCT. It represents a 45 cm diameter and a 24 cm axial length cooperatively with the patient that can be imaged with a single 360 degrees rotation of the gantry (103). The entire gantry (103) has been supported with base structure (104) along with +15 degrees or −15 degrees tilting motion. The patient couch table (105) has been mounted on the floor based on the gantry (103) reference plane. In the fan beam CT, one rotation of the gantry (103) is possible to cover small field (nearly 10 mm) of view only but the FPCBCT used in the present invention is able to cover higher FOV. Some other Flat Panel CT machines are also used but the FOV achieved is less compared to FOV obtained using FPCBCT.

Thus, the system (100) of the present invention performs enhancement of the image quality of a subject with less radiation exposure and high spatial resolution using CBCT.

The description of the present system has been presented for purposes of illustration and description but is not intended to be exhaustive or limited to the invention in the form disclosed. Many modifications and variations will be apparent to those of ordinary skill in the art without departing from the scope and 20 spirit of the invention. The embodiment was chosen and described in order to best explain the principles of the invention and the practical application, and to enable others of ordinary skill in the art to understand the invention for various embodiments with various modifications as are suited to the particular use contemplated.

We claim:

1. A system for enhancing image quality of a subject using CBCT (Cone Beam Computed Tomography), the system comprising:
a) An X-Ray Tube (102) configured to precisely adjust to reduce the radiation penumbra, wherein the X-Ray Tube (102) is mounted on a rotatable gantry (103), the rotatable gantry (103) has circular ring which is mechanically stabled with integrated imaging unit, the rotatable gantry (103) is supported with a base structure (104) along with an angle of 15 degrees or an angle of −15 degrees tilting motion, wherein the continuous rotation of the rotatable gantry (103) allow to capture of views which is difficult at zero degree position, wherein a patient couch table (105) is configured to scan which is mounted on the floor based on the rotatable gantry (103) reference plane; and
b) a plurality of movable flat panel imager (101) mounted on the opposite side of the X-Ray Tube (102), wherein the flat panel imager (101) is used along with the Cone Beam Computed Tomography (CBCT) to obtain superior image quality.

2. The system as claimed in claim 1, wherein each projection on the movable flat panel imager (101) is configured to image all the organs of a patient body in a single rotation.

3. The system as claimed in claim 1, wherein the greater Field of View (FOV) of the subject is obtained using a Flat Panel Cone Beam Computed Tomography (FPCBCT), wherein the FPBCT represents 45 cm diameter and a 24 cm axial length cooperatively with the patient is imaged with a single 360 degrees rotation of the gantry (103).

4. The system as claimed in claim 3, wherein the FPCBCT identifies soft tissue targets and the images obtained has reduced metal and beam hardening artifacts.

5. The system as claimed in claim 1, wherein the CBCT scanners has a two-dimensional detector which allows a single rotation of the x-ray source detector system to generate a scan of all the region of interest of patient body, wherein the images obtained using CBCT technique cover large volumes and has higher spatial resolution.
